(19) United States
(12) Patent Application Publication
(10) Pub. No.: US 2004/0086138 A1
(43) Pub. Date: May 6, 2004

(54) Ear protection and method for operating a noise-emitting device

(76) Inventor: Rainer Kuth, Herzogenaurach (DE)

Correspondence Address: SCHIFF HARDIN, LLP, PATENT DEPARTMENT, 6600 SEARS TOWER, CHICAGO, IL, 60606-6473, US

(21) Appl. No.: 10/471,887

(22) PCT Filed: Mar. 5, 2002
(86) PCT No.: PCT/DE02/00793

(30) Foreign Application Priority Data
Mar. 14, 2001 (DE) 10112305.1

(51) Int. Cl. A61F 11/06; G10K 11/16; H03B 29/00

(52) U.S. Cl. 381/72; 381/71.6

(57) ABSTRACT

An ear protection comprises a monitoring device, which is designed in order to be able to continuously monitor the effectiveness of the ear protection during use. The invention provides that, in a method for operating a noise-emitting device,during which at least one individual is wearing an ear protection and located in the area in which the noise-emitting device is generating noise, the noise-emitting device, in the event of a monitoring result of the ear protection indicating a risk of damage to the individual's hearing, is controlled whereby appropriately reducing the noise emission thereof.

### BACKGROUND OF THE INVENTION

1. Field of the Invention

The present invention is directed to a hearing protection as well as to a method for operating a noise-emitting device having a region in which noise produced by the device is present wherein at least one person with the hearing protection is situated.

2. Description of the Prior Art

A magnetic resonance apparatus in the scanning (data acquisition) mode, for example, represents a noise-emitting device with at least some of the noise having extremely high acoustic pressure levels. In a magnetic resonance apparatus, rapidly switched gradient fields that are generated by a gradient coil system are superimposed on a static basic magnetic field that is generated by a basic field magnet. The magnetic resonance apparatus also has a radiofrequency system that emits radiofrequency signals into the examination subject for triggering magnetic resonance signals and that picks up the magnetic resonance signals that have been triggered on the basis of which magnetic resonance images are produced.

For generating gradient fields, appropriate currents must be set in gradient coils of the gradient coil system. The amplitudes of the required currents amount to several 100 A. The current rise and decay rates amount to several 100 kA/s. Given a basic magnetic field on the order of magnitude of 1 Tesla, Lorentz forces that lead to oscillations of the gradient coil system act on these time-variable currents in the gradient coils. These oscillations are transmitted to the surface of the device via various propagation paths. These mechanical oscillations are converted thereat into acoustic oscillations that ultimately lead to undesirable noise emission.

The problem of noise emission has intensified as a consequence of the greatly enhanced performance capability of the gradient coil systems in recent years, particularly in combination with intensities of the basic magnetic field that have likewise increased. In modern high-performance magnetic resonance devices, the noise emissions reach peak values of approximately 140 dB. It is therefore recommended that a patient wear a double hearing protection composed of hearing protection plugs and a headphone-like hearing protection during an examination in the magnetic resonance apparatus.

The hearing protection plug is manually elastically tapered before introduction into the outer auditory canal and is thus pushed into the auditory canal. Therein, the hearing protection plug elastically expands, so that approximately 30 dB of externally occurring acoustic pressure levels are typically attenuated by the hearing protection plug.

At the beginning of an examination, a patient having normal reactions is given a pushbutton to hold, to allow the patient to signal the occurrence of a problem during the examination to an operator of the magnetic resonance apparatus by actuation of the pushbutton. When, for whatever reasons, the patient senses that the noises acting on him/her during, for example, the examination lie above a hearing-damaging level, then the patient can actuate the pushbutton. Damage to the hearing of the patient may already have occurred by the time the pushbutton is actuated. The risk of hearing damage is further intensified in case of sedated patients, who are not capable of actuating the pushbutton.

### SUMMARY OF THE INVENTION

An object of the invention is to provide an improved hearing protection and an improved method for the operation of a noise-emitting device with which—among other things—hearing damage can be reliably suppressed.

This object is achieved in a hearing protection having a monitoring device that is fashioned such that, given application of the hearing protection, the effectiveness of the hearing protection is continuously monitored. An acoustic pressure level that acts on a person protected by the hearing protection is directly or indirectly known at all times, so that, for example given an upward transgression of a prescribable limit value, corresponding counter-measures can be initiated, preferably automatically, so that damage to the person's hearing can be reliably precluded. The continuous monitoring need not be uninterrupted in time. The continuous monitoring can be implemented with digital or partially digital embodiment of the monitoring device that, as known, are based on a sampling of a temporally continuous signal and/or can be implemented with a method based on acquiring changes.

In an embodiment, the monitoring device has an output unit that emits a control signal. Given acoustic pressure levels that are too high, the control signal can deactivate the source causing the acoustic pressure levels or throttle it with respect to its noise emissions.

### DESCRIPTION OF THE DRAWINGS

FIG. 1 is a coronal section through a human head in the region of the outer auditory canal.

FIG. 2 is a sectional view of a first embodiment of a hearing protection plug in accordance with the invention, with a hydrostatic pressure sensor.

FIG. 3 is a sectional view of a second embodiment of a hearing protection plug in accordance with the invention, with a pressure sensor.

FIG. 4 is a sectional view of a third embodiment of a hearing protection plug in accordance with the invention, with a microphone.

FIG. 5 illustrates a first embodiment of a hearing protection module in accordance with the invention, with an arrangement for arranging an under-pressure.

FIG. 6 illustrates a second embodiment of a hearing protection module in accordance with the invention, with a transmission unit.

FIG. 7 is a schematic illustration of the basic components of a magnetic resonance apparatus operating in accordance with the invention.

### DESCRIPTION OF THE PREFERRED EMBODIMENTS

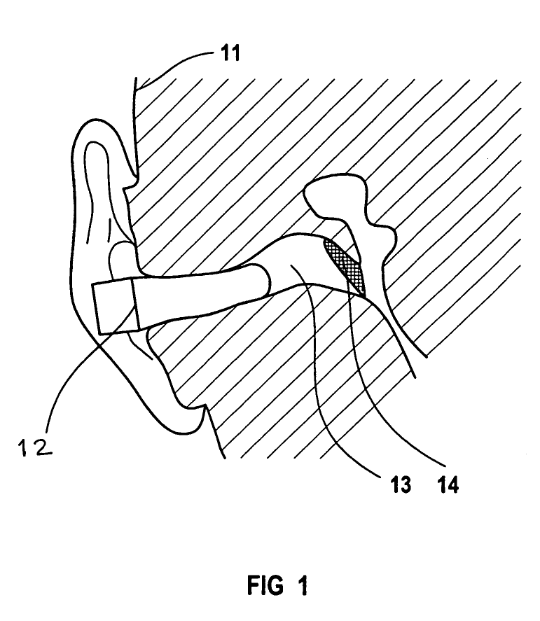

FIG. 1 shows an excerpt of a coronal section through a human head 11 in the region of the outer auditory canal 13. At one side, the outer auditory canal 13 is limited by the tympanic membrane 14. Further, a hearing protection plug 12 has been introduced into the outer auditory canal 13.

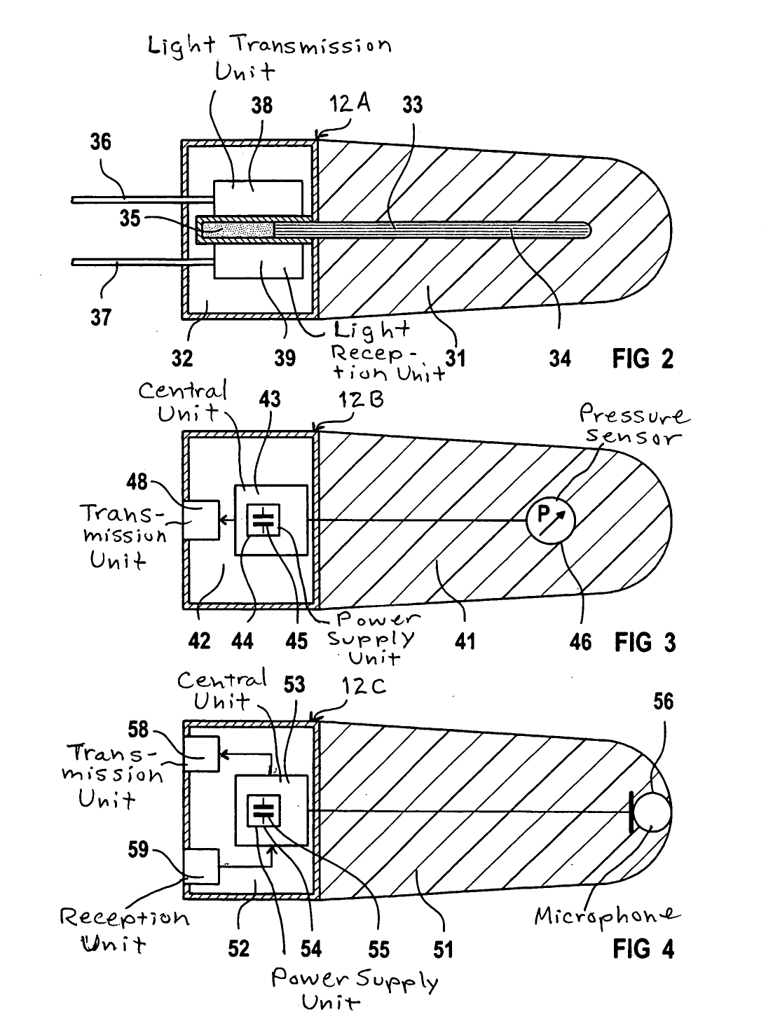

FIG. 2 shows a hearing protection plug 12A with a hydrostatic pressure sensor as a first exemplary embodiment of the invention. The hearing protection plug 12A has an elastically deformable inner part 31, an outer part 30 and an oblong cavity 33 that extends through the inner part 31 and the outer part 32. At a side facing toward the inner part 31, the cavity 33 is filled with, preferably, a dark-colored fluid 34, and the cavity 33 forms a reservoir for a preferably transparent gas 35 at a side facing toward the outer part 32. In one embodiment, the gas 35 is separated from the fluid 34 by a flexible membrane. A light transmission unit 38 as well as a light reception unit 39 are arranged in the region of the outer part 32 immediately adjacent to the cavity 33. The light transmission unit 38 and the light reception unit 39 are connected to respective light waveguides 36 and 37 for supplying a light signal and for conducting a light signal conducting.

Upon introduction of the hearing plug 12A into the outer auditory canal 13, the inner part 31 and, thus, the cavity 33 in the region of the inner part 31 are deformed, resulting in the boundary line between the fluid 34 and the gas 35 shifting dependent on the degree of deformation. A large shift of the boundary line in the direction of the outer part 32 results from a high pressure on the inner part 31, which indicates a high pressing force of the hearing protection plug 12A inside the outer auditory canal 31 and indicates a good noise-damping effect of the hearing protection plug 12A. The aforementioned information is output via the light signal of the light waveguide 37 connected to the light reception unit 39 and, for example, can be employed for controlling a device that emits the noises to be damped by the hearing protection plug 12A.

In one embodiment, the light signal is emitted at only two signal levels—for example, light on and light off—for indicating whether the pressure lies above or below a prescribable limit value. In another embodiment, the light signal is output with a changing intensity that continuously represents a measure of the pressure. Dependent on the position of the boundary line, more or less light from the light transmission unit 38 is transmitted to the light reception unit 39, a low intensity thus corresponding to a high pressure onto the inner part 31. An evaluation device (not shown) connected to the light waveguide 37 continuously converts the intensity of the light signal into an indication of the effectiveness of the hearing protection plug 30 to attenuate the acoustic pressure level.

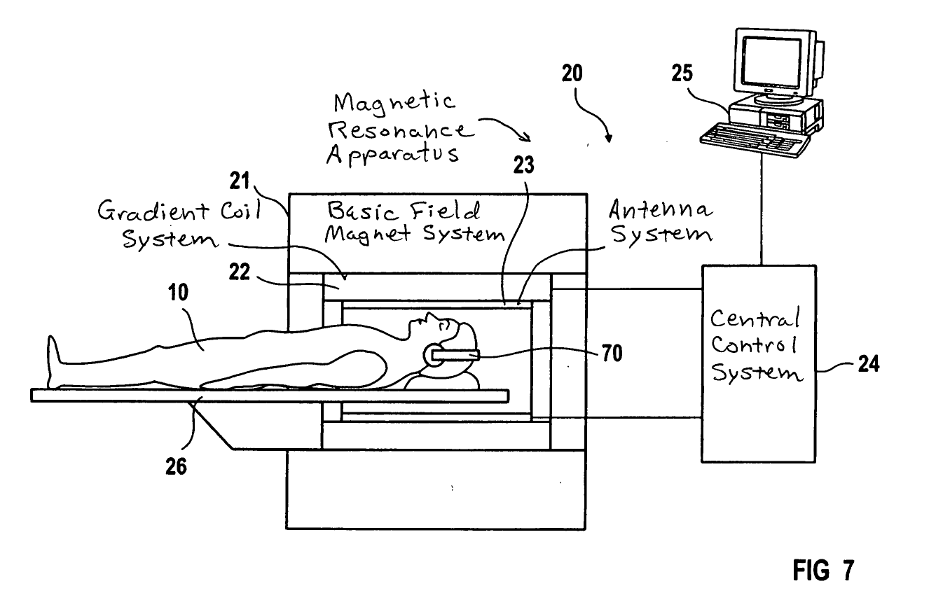

As a result of its hydrostatic pressure conversion and the purely optical pressure detection based thereon, the hearing protection plug 12A of FIG. 2 can be fashioned free of metallic, particularly ferro-magnetic, component parts in a simple way, so the hearing protection plug 12A can be unproblematically utilized within a magnetic resonance apparatus 20 (see FIG. 7) with an optimally high electromagnetic compatibility.

In a second exemplary embodiment of the invention, FIG. 3 shows a lead-free hearing protection plug 12B with a pressure sensor 46. The pressure sensor 46 is thereby arranged such in the inner part of the hearing protection plug 12B so that it normally comes to lie just barely inside the outer auditory canal 13 after introduction of the hearing protection plug 12B. For example, the pressure sensor 46 can be fashioned such that it continuously converts the pressure acting on it into a corresponding signal. The pressure sensor 46 is connected to a central unit 43 arranged in the outer part 42 of the hearing protection plug 40 among other things, for forwarding the signal. Further, the central unit 43 is connected to a transmission unit 48 for non-hardwired transmission of information that is likewise arranged in the outer part 43. The transmission unit 48 is fashioned, for example, as an infrared or microwave transmission unit. For the energy supply of the central unit 43, of the pressure sensor 46 as well as of the transmission unit 48, the central unit 43 contains an energy supply unit 44 having a double-film capacitor 45 with high capacitance and high power density. German 199 35 915 A1—which is incorporated herein by reference —provides a more detailed description of the energy supply unit 44 and of the transmission unit 48.

In one embodiment, a continuous evaluation of the signal of the pressure sensor 46 ensues in the central unit 43 in order to determine whether the pressure lies above or below a prescribable limit value, so that the transmission unit 48, for sending a signal, in conformity with the evaluation, need only have two signal states that are distinguishable from one another. In another embodiment, the values of the pressure acquired by the pressure sensor 46 are continuously sent with an appropriately encoded signal to an evaluation device (not shown) that is arranged remote from the hearing protection plug 40. The signal of the pressure sensor 46 is correspondingly edited in the central unit 43 for transmission by the transmission unit 48.

Given the fashioning of the transmission unit 48 as microwave transmission unit and employment of the hearing protection capsule 40 in or at a magnetic resonance apparatus 20, care must be exercised to see that a transmission frequency of the microwave transmission unit lies above a nuclear magnetic resonance frequency of the magnetic resonance apparatus 20, particularly above 100 MHz. Harmonics of the transmitted signal can thus also not cause interferences with the nuclear magnetic resonance frequency. The nuclear magnetic resonance frequency, which is proportional to a basic magnetic field strength, amounts to approximately 84 MHz given a basic magnetic field strength of, for example, 2 Tesla. Care must also be exercised in the selection of the transmission frequency that this is respectively approved by the appertaining authorities. In Germany, for example, the transmission frequency of 433.92 MHz is approved.

As a third exemplary embodiment of the invention, FIG. 4 shows a hearing protection plug 12C with a microphone 56. For the continuous detection of an acoustic pressure level acting on the tympanic membrane 14, the microphone 56 is arranged directly at that side of an inner part 51 of the hearing protection plug 12C that faces toward the tympanic membrane 14. For, among other things, forwarding the acoustic pressure level acquired by the microphone 56, the microphone 56 is connected to a central unit 53 arranged in an outer part 52 of the hearing protection plug 12C. Further, the central unit 53 is connected to a transmission unit 58 and reception unit 59 likewise arranged in the outer part 52 for the non-hardwired transmission and reception of information. For the energy supply of the central unit 53, of the microphone 56 as well as of the transmission and reception unit 58 and 59, the central unit 53 contains an energy supply unit 54 with a double-film capacitor 55 having high capacitance and high power density. The description pertaining to the embodiment of FIG. 3 applies to the further design and operation of the microphone 56, of the central unit 53 (including its power supply unit 54), and the transmission unit 58. Via the reception unit 59, further, it is possible to control operation of the hearing protection plug 12C, particularly operation of the central unit 53. In one embodiment wherein a speaker (not shown) is arranged in addition to the microphone 56, an externally controllable output of tones, voice message and/or prospective anti-sound can be realized.

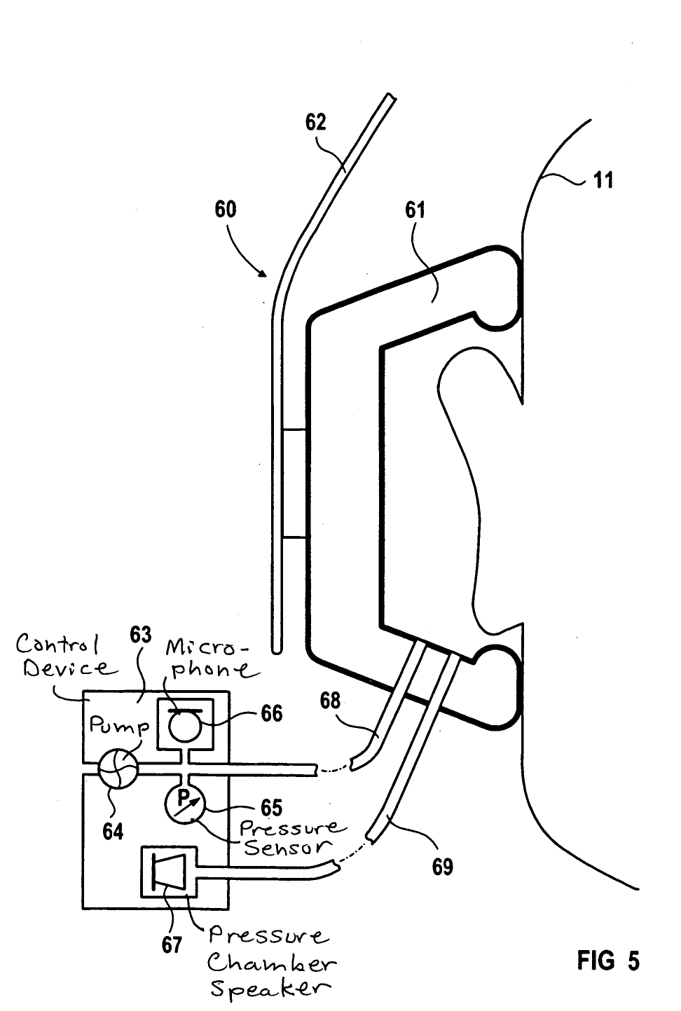

As a fourth exemplary embodiment of the invention, FIG. 5 shows one half of a coronary section through a human head 11 with a hearing protection module 61. For forming a hearing protection 60 similar to headphones, the hearing protection module 61 is connected to a further hearing protection module (not shown) by a connector 62. The hearing protection module 61 is connected to a first and a second lead conduits 68 and 69 with which the space formed by the head 11 and the hearing protection module 61 is connected to a control device 63 at a distance from the hearing protection.

The first lead conduit 68 is connected to a pump 64 in the control device 63 for producing an under-pressure within the space. Further, the space is connected via the first lead conduit 68 to a gas pressure sensor 65 and a microphone 66. A measurement of the under-pressure within the space and, dependent on a corresponding drive of the pump 64 can be implemented via the gas pressure sensor 65. An under-pressure of, for example, 200 mbar that a person still finds to be pleasant is thereby set. The under-pressure effects a good seating of the hearing protection conduit 61 at the head 11. A frequent actuation of the pump 64 for maintaining the under-pressure thereby indicates a poor fit of the hearing protection capsule 61 at the head 11. An acoustic pressure level within the space can be continuously monitored with the microphone 66.

The second lead conduit 69 is connected to a pressure chamber speaker 67 in the control device 63. With an appropriate drive, the pressure chamber speaker 67 can be used, for example, for producing prospective anti-noise within the space. Given employment of the hearing protection 60 in or at a magnetic resonance apparatus 20, a characteristic pattern with reduced amplitude values that repeats within the examination sequence is implemented once for the sequence to be implemented. The noises resulting therefrom are recorded and correspondingly employed for the control of the prospective anti-noise per repetition of the characteristic pattern upon implementation of the sequence.

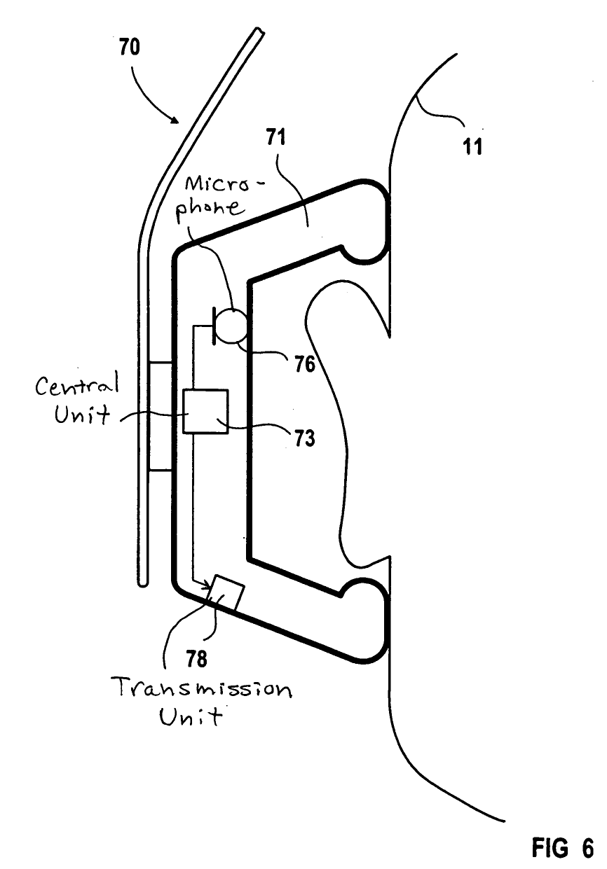

As a fifth exemplary embodiment of the invention, FIG. 6 shows a hearing protection module 71 of a hearing protection 70 for a human head 11 fashioned like a headset. For the continuous monitoring of an acoustic pressure level within the space formed between the head 11 and the inside of the hearing protection module 71, the hearing protection module 71 has a microphone 76, a central unit 73 (including an energy supply unit 74), and a transmission unit 78. The description pertaining to the embodiment of FIG. 4 applies for the units 73 through 78 as well as their functioning.

It should be noted that the relative damping effect of the hearing protection plugs 12A and 12B on the acoustic pressure level is only indirectly monitored via their pressing force in the outer auditory canal 13, and the absolute acoustic pressure level occurring at the tympanic membrane 14 is not monitored. In the hearing protections 50, 60 and 70, in contrast, the absolute acoustic pressure level occurring at the tympanic membrane 14 can be monitored.

FIG. 7 is a schematic illustration of a magnetic resonance apparatus 20. The magnetic resonance apparatus 20 thereby has a basic field magnet system 21 for generating a basic magnetic field and a gradient coil system 22 for generating gradient fields. The magnetic resonance apparatus 20 has an antenna system 23 for emitting radiofrequency signals in as well as for acquiring the magnetic resonance signals generated as a result thereof. The gradient coil system 22 is connected to a central control system 24 for controlling currents in the gradient coil system 22 on the basis of a selected sequence. The antenna system 23 is likewise connected to the central control system 24 for controlling the radiofrequency signals to be emitted according to the selected sequence as well as for the further-processing and storing of the magnetic resonance signals acquired by the antenna system 23. For, among other things, positioning a region of a patient 10 under examination to be imaged in the magnetic resonance apparatus 20, the magnetic resonance apparatus 20 has a movable support mechanism 26 on which the patient 10 is placed. The central control system 24 is connected to a display and operating device 25 via which inputs of an operator, for example the desired sequence type and sequence parameters, are supplied to the central control system 24. Among other things, further, the generated magnetic resonance images are displayed at the display and operating device 25.

The patient 10 on the support mechanism 26 wears, for example, the hearing protection 70 according to FIG. 6. The central control system 24 of the magnetic resonance apparatus 20 is fashioned such that it receives information about the acoustic pressure level at the tympanic membranes 14 of the patient 10 continuously transmitted from the hearing protection 70, and can automatically control or abort an ongoing sequence such that an acoustic pressure level at the tympanic membranes 14 of the patient 10 does not exceed a prescribable limit value of, for example, 80 dBA. Damage to the hearing of the patient 10 is thus reliably precluded. This also applies to sedated patients.

Although modifications and changes may be suggested by those skilled in the art, it is the invention of the inventor to embody within the patent warranted heron all changes and modifications as reasonably and properly come within the scope of his contribution to the art.

1. Hearing protection, whereby the hearing protection comprises a monitoring device that is fashioned such that the effectiveness of the hearing protection can be continuously monitored given employment of said hearing protection.

2. Hearing protection according to claim 1, whereby the monitoring device comprises means for the acquisition of an acoustic pressure level.

3. Hearing protection according to claim 2, whereby the means are arranged such that an acoustic pressure level effective at a tympanic membrane can be acquired.

4. Hearing protection according to one of the claims 2 or 3, whereby the means comprise a microphone.

5. Hearing protection according to one of the claims 2 through 4, whereby the monitoring device comprises means with which the acoustic pressure level can be monitored in view of an upward transgression of a prescribable limit value.

6. Hearing protection according to one of the claims 1 through 5, whereby the monitoring device comprises means for the output of a control signal.

7. Hearing protection according to one of the claims 1 through 6, whereby the monitoring device comprises an energy supply device, preferably containing a double-film capacitor.

8. Hearing protection according to one of the claims 1 through 7, whereby the hearing protection is fashioned free of leads.

9. Hearing protection according to one of the claims 1 through 8, whereby the hearing protection is fashioned free of metallic and/to ferromagnetic materials.

10. Hearing protection according to one of the claims 1 through 9, whereby the hearing protection comprises an elastic part for the introduction into an auditory canal.

11. Hearing protection according to claim 10, whereby at least one part of the monitoring device is arranged in the elastic part.

12. Hearing protection according to claim 11, whereby the part of the monitoring device comprises means for acquiring a deformation of the elastic part.

13. Hearing protection according to claim 12, whereby the means comprise a pressure sensor.

14. Hearing protection according to one of the claims 1 through 13, whereby the hearing protection comprised a hearing protection capsule for covering an auditory canal opening.

15. Hearing protection according to claim 14, whereby the hearing protection comprises means for generating and/or monitoring an under-pressure in a space covered by the hearing protection capsule.

16. Hearing protection according to one of the claims 14 or 15, whereby the hearing protection capsule is part of a hearing protection fashioned like a headset.

17. Method for the operation of a noise-emitting device in whose range of noise influence at least one person with a hearing protection according to one of the claim 1 through 16 is situated, whereby, given a monitoring result of the hearing protection that there is a risk of damage to the hearing of the person, the noise-emitting device is controlled to the effect that its noise emission is correspondingly reduced.

18. Method according to claim 17, whereby the noise-emitting device is a magnetic resonance apparatus.

19. Method according to one of the claims 17 or 18, whereby the person is a patient.
